(19) United States
(12) Patent Application Publication
(10) Pub. No.: US 2022/0256842 A1
(43) Pub. Date: Aug. 18, 2022

(54) NEW FORMULATION BASED ON AN OLEO GEL, IN PARTICULAR FOR RELEASING VOLATILE COMPONENTS, AND METHOD FOR THE PRODUCTION THEREOF

(71) Applicant: FACHHOCHSCHULE BIELEFELD, Bielefeld (DE)

(72) Inventors: Anant PATEL, Bielefeld (DE); Linda MUSKAT, Bielefeld (DE); Pascal HUMBERT, Gütersloh (DE)

(21) Appl. No.: 17/628,741

(22) PCT Filed: Jul. 23, 2020
(86) PCT No.: PCT/EP2020/070763

§ 371 Date: Jan. 20, 2022

(30) Foreign Application Priority Data
Jul. 23, 2019 (DE) 10 2019 119 888.7

(51) Int. Cl. A01N 25/04

(52) U.S. Cl. CPC A01N 25/04

(57) ABSTRACT

In a first embodiment, the invention relates to an oleo gel formulation suitable for releasing volatile components, such as semiochemicals, said formulation containing at least one oleo gelator, such as alkyl cellulose, a volatile component and a non-polar fluid. In another embodiment, an oleo gel formulation is provided, containing an active ingredient including at least one gelator, the active ingredient and a non-polar fluid. The invention further relates to a method for producing the oleo gel formulation according to the invention. Finally, the invention relates to the use of the oleo gel formulation according to the invention, in particular as a plant protection agent and pesticide, but also in the food or pharmaceutical industry containing pharmaceutical or cosmetic active substances, flavours or perfumes.

In a first aspect the present invention is directed to an oleogel formulation suitable for releasing volatile components, such as semiochemicals; this formulation comprises at least one oleogelator, such as alkylcellulose, a volatile component, and a nonpolar fluid. In a further aspect an oleogel formulation is provided that contains an active ingredient comprising at least one oleogelator, the active ingredient, and a nonpolar fluid. The present invention further relates to a method for producing the oleogel formulation of the invention. Described lastly is the use of the oleogel formulation of the invention more particularly as a crop protection product and pesticide or alternatively in the food industry or the pharmaceutical industry, containing active pharmaceutical or cosmetic ingredients, flavors or fragrances.

### PRIOR ART

In the provision of components and active ingredients a major problem is the appropriate formulation of these components and ingredients in appropriate formulations. Different formulations are needed according to the range of requirements. Such ingredients and components are frequently provided in formulations which allow these components and ingredients to be released at a certain point in time or over a prolonged period. Such formulations find application not only in the pharmaceutical and cosmetic sectors but also in the food sector. Other sectors include agriculture, with examples being crop protection products and pesticides.

Around 80% of all formulation problems arise from the formulation of hydrophilic or hydrophobic molecules, examples being active pharmaceutical and cosmetic ingredients, crop protection agents and trace elements.

Relatively large hydrophilic molecules, such as enzymes, for example, can be enclosed and provided in ready-to-use form by inclusion in conventional hydrogels, composed of alginate or pectinate, for example. For small hydrophilic molecules in particular there is a lack of suitable formulation technologies and materials. In recent decades in particular there has been increased development of active ingredients which are prepared in aqueous syntheses, and consequently are hydrophilic in nature. Examples of such active ingredients include small therapeutic proteins, RNAi, biological and chemical crop protection agents, water-soluble vitamins, trace elements, and other active pharmaceutical and cosmetic ingredients.

The microencapsulation of pesticides, in the crop protection sector, for example, has been known for some considerable time. WO 90/00005, for instance, describes microencapsulated pesticides with an attractant. This attractant is present in an outer shell with an outer casing, while the core of this capsule contains a pesticide. In this system the insect is attracted by the attractant and the insect consumes a pesticide when it pierces the capsule.

DE 10 2015 016 114 A1 discloses liquid-core capsules for controlling pests such as biting-sucking insects. This liquid-core capsule has an aqueous core and a diffusion-inhibiting outer shell. While the outer shell contains an attractant for the pest, the core contains a pesticide. This document describes the use of hydrogel shells as outer material. Similar constructions are found in EP 2 415 356 A2. Described here are capsules with a solid shell, the shell comprising a diffusion barrier layer composed, for example, of ethylcellulose and/or resin-based polymers. WO 2015/073439 A1 describes liposomal attractant formulations comprising pesticides or nematicides. This is a liposomal formulation, where the pesticides or nematicides reside in the aqueous core of liposomes and where one or more attractants for the pest or the nematodes are present in the lipid bilayer or are bound on the surface of the liposomes.

Oleogels have recently been garnering increased attention especially in the sector of active pharmaceutical, food-technology and cosmetic ingredients; they have not to date been used in agriculture. According to the definition in the European Pharmacopeia, gels are gelled liquids produced using suitable swelling agents. A distinction is made between hydrophilic gels (hydrogels) and lipophilic gels (oleogels). While the bases of hydrophilic gels are typically water, glycerol or propylene glycol with suitable swelling compounds such as poloxamers, starch, cellulose derivatives, carbomers or silicates, lipophilic gels are frequently preparations in which liquid paraffin is gelled with polyethylene or fatty oils are gelled with colloidal silicon dioxide, aluminum soaps or zinc soaps. These gelling agents are also described as oleogelators. Zetzl A. K. et al., Food Structure 2, 2014, 27-40 describe the mechanical properties and the microstructure of ethylcellulose oleogels. Also described therein are a multiplicity of suitable oleogelators which can be classified on the basis of their properties: formation of the network of crystalline particles, the self-assembling fibrillar networks, and polymer gelation. Corresponding oleogelators are described in this document, and include waxes, monoacylglycerides, ceramides, and also lecithin, etc. Typical formulas include a small fraction of ethylcellulose as oleogelator and corresponding liquids to be gelled, such as vegetable oils, etc.

One problem of conventional hydrogel capsules is an early drying-up, which is to be avoided. Furthermore, there is a demand for new formulations which are able to release the molecules or components and ingredients in a desired way and at the same time are able to accommodate corresponding fractions of these ingredients and molecules, and which also, furthermore, satisfy the heightened environmental requirements.

### BRIEF DESCRIPTION OF THE INVENTION

The invention provides an innovative formulation which is able to release volatile components and/or other active ingredients. Small hydrophilic components in particular can be formulated with the innovative capsule formulation in such a way that they are available in a desired manner. This includes use not only in the crop protection sector and agriculture as a whole, but also in the food, pharmaceutical, and cosmetic sectors.

Provided in a first aspect here is an oleogel formulation suitable for releasing volatile components, such as semiochemicals, comprising:
0.1-20 wt % of oleogelator, such as alkylcellulose;
at least 0.1 wt % of the volatile component, such as volatile semiochemicals; and
nonpolar fluid ad 100 wt %, where the volatile component may be part of the nonpolar fluid.

Provided in a further aspect is an oleogel formulation containing an active ingredient, comprising:
0.1-20 wt % of oleogelator, such as alkylcellulose;
at least 0.1 wt % of active ingredient, optionally in solution in a hydrophilic liquid; and
a nonpolar fluid ad 100 wt %.

It has emerged that the capsule shell of oleogel exhibits advantages both over the bilayer structure of liposomes and over hydrogels as hydrophilic gels. In particular, hydrophilic ingredients and components can be provided with the formulation of the invention.

Provided in a further aspect is a method for producing the oleogel formulation of the invention; this method comprises:
providing the nonpolar fluid;
introducing the oleogelator into the liquid nonpolar fluid, to give a liquid mixture containing oleogelator and nonpolar fluid; and
producing the formulation, where the volatile component or the active ingredients are added, depending on the hydrophobicity, to the nonpolar fluid and to the gelator before introduction into the liquid nonpolar fluid, or as a further solution separately.
   The present patent application is directed, finally, to the use of the formulations of the invention as crop protection products and pesticides, more particularly containing a biocide, insecticide, fungicide, nematicide, acaricide and/or herbicide, and also in the food, cosmetic or pharmaceutical industry.

### DESCRIPTION OF THE FIGURES

FIG. 1 illustrates various types of formulation.

### DETAILED DESCRIPTION OF THE PRESENT INVENTION

The invention relates to a formulation based on an oleogel, i.e., to an oleogel formulation. This formulation is suitable for releasing volatile components and/or active ingredients. In one embodiment the oleogel formulation is suitable for releasing volatile components, such as semiochemicals, comprising:
0.1-20 wt % of oleogelator, such as alkylcellulose;
at least 0.1 wt % of the volatile component, such as volatile semiochemicals; and
nonpolar fluid ad 100 wt %, where the volatile component may be part of the nonpolar fluid.

Provided in a further aspect is an oleogel formulation containing an active ingredient, comprising
0.1-20 wt % of oleogelator, such as alkylcellulose;
at least 0.1 wt % of active ingredient, optionally in solution in a hydrophilic liquid; and
a nonpolar fluid ad 100 wt %.

The inventors have been able to show that with the formulation of the invention it is possible to provide not only hydrophilic components and ingredients but also hydrophobic components and ingredients, and especially volatile components such as volatile semiochemicals, in the formulation. Through the formulation of the invention it is possible for the volatile semiochemicals or other volatile components to be delivered, and/or the active ingredients provided, to mandated extent and at mandated times. Relative to conventional hydrogel capsules, the formulations of the invention exhibit advantages in the formulation of hydrophilic components, and also with respect to drying up.

In the invention the oleogel here is formed of the nonpolar fluid and the oleogelator.

Depending on the nature of the provision, the oleogelator is used in an amount of 0.1-20 wt %. Small amounts such as 0.1-5 wt % allow the provision of relatively pastelike formulations; larger amounts such as 2-20 wt %, e.g. 5-20 wt %, such as 7-15 wt %, allow the provision of oleogels with stronger gelling, in the form of capsules, etc., for example. Where ethylcellulose is a constituent of the oleogelator, amounts of 10 to 20 wt % are used, for example; with waxes, smaller amounts are sufficient, such as amounts in the range from 0.1 to 4 wt %.

In the present invention the expression “oleogelator” is understood to refer to the following: molecules which by formation of three-dimensional structures bring about an increase in the viscosity of a nonpolar fluid. In other words, these molecules bring about gelling of the nonpolar fluid, with an increase in the viscosity. An oleogelator is the gel-former of an oleogel. Unless otherwise observed, the oleogelator may be one oleogelator or a combination of at least two oleogelators.

The expression “oleogel” is presently understood to refer to a nonpolar, customarily lipid fluid which is solidified by gelling with an oleogelator.

The expression “volatile component” is presently understood to mean that the components, such as the ingredient or the molecules, are volatile at a temperature of 20° C. (room temperature). These are substances which readily evaporate or are already present in gas form at low temperatures and under atmospheric pressure. Depending on application, these substances may only be volatile at their intended locations, for example under certain temperature conditions following application in agricultural use or on the body in the case of pharmaceutical or cosmetic applications, in other words at temperatures of 30° C. or more.

The expression “nonpolar fluid” is presently understood to refer to a fluid where the molecules have no permanent dipole properties, meaning that the charge distribution in the molecule is largely uniform. Nonpolar compounds dissolve well in nonpolar solvents (e.g., benzene), but not in polar solvents such as water.

The expression “active ingredient” is understood presently to refer to a component or a molecule which at the intended location is responsible for the activity. In the case of pharmaceutical or cosmetic uses, these are compounds which exhibit an activity at the location of effect; in the case of foods, they are vitamins or aromas, for example, and in the case of crop protection products or pesticides they are the molecules which have an effect on the pests or which protect the plants, including attractants, nutrients and pesticides, biocides, insecticides, nematicides, acaricides, herbicides or fungicides.

The expressions “comprising” or “containing” include the embodiments of “consisting of”. Unless otherwise observed, the expressions “a”, “an” or “one” or “the” encompass “one or more” of the stated constituents.

In one embodiment the oleogelator is at least one oleogelator selected from alkylcellulose, wax, more particularly vegetable wax, sterols, fatty alcohols, fatty acids, wax acid, wax esters, fatty acid esters, isoprene derivatives, mono-, di- and/or triglycerides, lecithins, stearates, proteins, lignins, keratins, polysaccharides such as chitin derivatives, etc. The oleogelator here may comprise at least one component. In one embodiment there are at least two oleogelators.

In one embodiment the alkylcellulose is more particularly an ethylcellulose. In the case of a combination of oleogelators, one embodiment is the use of alkylcellulose, ethylcellulose for example, in combination with a vegetable wax. In one preferred embodiment the combination is one of ethylcellulose and carnauba wax or a combination of ethylcellulose with candelilla wax. Other preferred vegetable waxes are berry wax, myrtle wax, bees wax, etc. These waxes are able to modulate the polarity and/or release. They may also be used, furthermore, for modulating the surface of the resulting gel formulations. Ethylcellulose and alkylcellulose in general are polymeric oleogelators, via which the corresponding gelling is controlled. The viscosity of the gel can be adjusted depending on the selected oleogelators and on the nonpolar fluids and on their proportions. The viscosity is increased, according to the development of the 3D network, up to a point of solidification of the gel.

In one embodiment the oleogelators are a combination of alkylcellulose and vegetable wax. Particularly suitable combinations are those of ethylcellulose with the vegetable wax, such as carnauba wax and candelilla wax, in which case the latter are present in amounts of 0.1-20% and ratios of 2:0.01 to 0.01:2.

Suitable nonpolar fluids include, in particular, those selected from vegetable, animal, microbial or mineral oils. Examples of suitable materials are sunflower oil, rapeseed oil, sesame oil, olive oil, linseed oil, maize germ oil, palm oil, soybean oil, coco butter, fish oil, liquid paraffin, castor oil, hazelnut oil, coconut oil, peanut oil, palm kernel oil, cottonseed oil, and also saturated, monounsaturated and polyunsaturated fatty acids (PUFAs), such as arachidonic acid, and fatty alcohols, such as octyldodecanol, or else co-solvents such as hexane, xylene, etc. Vegetable oils are preferred, especially those as stated above.

In one embodiment further adjuvants are added; this adjuvant may be at least one selected from particulate solids for delayed release, fillers, UV protectants, absorbers, density modifiers, temperature-dependent polymorphous or crystalline substances such as waxes, plasticizers such as octyldodecanol, and pigments. Adjuvants added may be, in particular, adjuvants which undergo temperature-dependent change, examples being amorphous, semicrystalline, polymorphous and/or crystalline substances such as the waxes or plasticizers such as octyldodecanol.

The oleogel formulation is more particularly one in which there is octyldodecanol; these gels exhibit particularly good processing properties at reduced process temperature.

Besides these adjuvants, there may also be, in particular, additional flavors, fragrances, attractants, etc.

In one embodiment the formulation is one in which the volatile component or the active ingredient is a hydrophilic component or a hydrophilic active ingredient. This hydrophilic component or the hydrophilic active ingredient is in solution, in one embodiment, in a polar solvent. Suitable polar solvents are, in particular, aqueous solvents which permit corresponding dissolution of the components and the molecules and ingredients in suitable concentrations.

Preferred hydrophilic components or active ingredients include semiochemicals, short-chain fatty acids, polysaccharides, sugars and sugar alcohols, nucleic acids, e.g., RNAi, amino acids, proteins, (phyto)hormones, parahormones, biocides/pesticides (insecticides, e.g., Bt toxin or viruses, nematicides, herbicides, fungicides, etc.), vitamins, alcohols, minerals, fertilizers, plant dyes, etc.

In a further embodiment the oleogel may comprise hydrophobic component and ingredients, such as volatile semiochemicals. These are located correspondingly in the gel formed from the oleogelator and the nonpolar fluid.

In one embodiment the semiochemicals are volatile pheromones or volatile allelochemicals (such as kairomones, allomones, synomones).

The expression “semiochemicals” is used generally to mean that these are compounds or molecules, known as messenger compounds, which serve for chemical communication between individual members of one species or between different species. The semiochemicals which can be used in the invention are, more particularly, chemicals which are released by an individual in order to alter or influence the behavior or metabolism of the other individual. Suitable semiochemicals include, in particular, pheromones, kairomones, allomones, synomones, hormones, phytohormones, parahormones, and (glycol) proteins.

One embodiment of the present invention is a formulation in the form of a core-shell capsule; this core-shell capsule customarily comprises a hydrophobic oleogel shell with a hydrophilic core.

The formulation in the form of a core-shell capsule may, for example, be one which has a liquid core, more particularly a liquid hydrophilic core, with a shell formed of hydrophobic oleogels. This means that the oleogel formulation of the invention is suitable for forming the shell of such core-shell capsules. These core-shell capsules of the invention are suitable more particularly for the provision of formulations with hydrophilic volatile components and/or with active ingredients, these active ingredients optionally being in solution in the hydrophilic liquid.

The skilled person is aware of corresponding methods for producing such core-shell capsules. For instance, the US application US 2009/004337 A1 describes the formation of core-shell capsules by coextrusion. In other words, a multi-fluid die, such as a two-fluid die or three-fluid die, etc., is used. Other possibilities for forming core-shell capsules are described in U.S. Pat. No. 3,856,699, for example.

The expressions “hydrophilic” and “polar” are used herein synonymously unless otherwise observed.

This embodiment of the core-shell capsule is suitable particularly in agriculture, in the form for example of sucking or biting capsules for sucking, piercing and biting insects, as a crop protection product or pesticide. It can be used in particular as a herbicide, fungicide, biocide or insecticide. For example, the capsules may serve, with a polar, hydrophilic liquid core, encased by the hydrophobic oleogel, for harmful insects, such as bugs, psyllids, aphids, etc., and also for useful ichneumon wasps. The capsules in this case comprise attractants, such as volatile semiochemicals, examples being volatile pheromones, which on the basis of the selected composition of the oleogel allows retarded and/or controllable release of these attractants for the purpose of manipulating the behavior of harmful and beneficial insects. These attractants may also take form of alarm substances, meaning that they have a repellent effect on the corresponding harmful and beneficial insects.

In one embodiment there are also additional components, ingredients or molecules present in one region, such as the hydrophilic core of the core-shell capsules, which permit feeding or killing of the harmful and beneficial insects. This means that the hydrophilic core contains corresponding biocides, insecticides, pesticides or herbicides. Alternatively for feeding there may be corresponding phagostimulants present. For example, there may be corresponding food substrates for the target insects, i.e., the harmful and/or beneficial insects. Examples of food substrates are those which mimic the natural food substrates. A composition thereof may imitate, for example, the composition of the phloem of the respective host plant. A nutrient substrate of this kind may consist, for example, of water and corresponding amino acids and sugars or sugar alcohol such as sorbitol. Optionally there may be additional constituents present as stabilizers, such as gelling alkylcellulose as a temporary stabilizer. In one embodiment the food substrate is present in an amount of 70 to 95 wt % of water and 30 wt % to 5 wt % of sugar or sugar alcohol; in the case of corresponding further constituents, these stated amounts are adapted to 100. Besides the compositions stated, there may also be corresponding amino acid mixtures included.

In this way the oleogel formulations of the invention can be designed in terms of their permeability and elasticity in such a way that they permit corresponding sucking, piercing or biting by the insects. This means that the oleogel is designed correspondingly to be capable of penetration by the proboscis or mouth organs of the insects.

For example, certain insects feed by sucking, and so during the sucking process they may transmit pathogens for plant diseases (e.g., phytoplasmosis) to plants via their saliva.

The formulations of the invention make it possible on the one hand to sample the saliva of these insects and on the other hand to kill the insects, from the consumption of formulated insecticides. The formulations more particularly are so-called attract-and-kill formulations, for attracting and subsequently killing plant pests.

In another embodiment of the present invention, the formulation may be in the form of capsules or granules with hydrophilic regions. These hydrophilic regions are surrounded by hydrophobic oleogel. A further embodiment is the provision of the formulation as a matrix, in the form of a sphere, for example, such a solid sphere or hollow sphere, or a fiber, block, lens or film. These stated embodiments may comprise the volatile component in the oleogel or surrounded by the oleogel in hydrophilic regions.

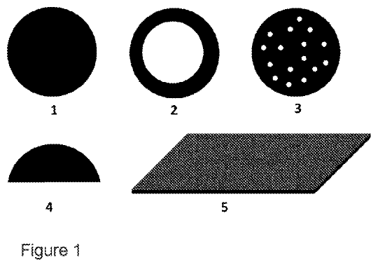

FIG. 1 represents various embodiments of the oleogel formulations. Embodiment 1 shows a sphere based on an oleogel matrix. The formulation of embodiment 2 shows a hollow sphere with a hydrophilic core, enclosed by a hydrophobic oleogel. Embodiment 3 is a formulation based on an oleogel matrix with a plurality of hydrophilic cores. The formulation of embodiment 4 shows a matrix which may take the form of embodiments 1 to 3, in the form of a hemisphere. Lastly, the embodiment shows a formulation such as a matrix, in accordance with embodiments 1 to 3, in the form of a film.

In one embodiment the formulation may be one containing hydrophilic active ingredients and/or hydrophilic volatile components in at least one hydrophilic region and hydrophobic active ingredients and/or hydrophobic volatile components in the hydrophobic region formed by the oleogel. This includes, in particular, the above-described core-shell capsules having an attractant and/or alarm substance typically in the hydrophobic region formed by the oleogel, and a further modifying constituent, such as a kill component or a food substrate, in the hydrophilic region, typically in the core region.

In one embodiment the formulation may be in the form of a paste. Such pastes are especially suitable for introduction into difficult-to-access regions, but also on plants, in barns, as an insect repellent carrier for human and livestock use, or for introduction into joints or pores for pest prevention. Here, optionally, a further gelling may be initiated after the paste has been applied.

The formulation in one embodiment is an attract-arrest-kill capsule, having an attractant in the shell, having nutrients (arrest) contained in the core, and having hydrophilic, biological or chemical insecticides, herbicides, pesticides, biocides, nematicides, acaricides or fungicides in the core. The killing component may optionally also be present on the surface of the capsule, or separately.

The formulation of the invention is one which can be produced simply, inexpensively and nontoxically. In certain embodiments it is based in particular on entirely renewable raw materials and is therefore biodegradable. This is especially the case for embodiments with nonpolar fluids comprising vegetable oils, and with oleogelators based on alkylcellulose, optionally with a wax, such as a vegetable wax, as a second component. As nonpolar fluid it is also possible here to use recycled vegetable oils, such as used frying fat, for example. The formulation of the invention permits universal capacity for use with different hydrophilic components. It can be used as a crop protection product and pesticide, or alternatively in the cosmetic or pharmaceutical sector, or else in the food sector. Because of the simple possibility of modifying the elasticity and permeability of the oleogel, a wide variety of different formulation properties can be adapted to the corresponding requirements. These corresponding properties, including thermoplastic properties of the formulation, enable biodegradable pheromone dispensers in various sizes and formats, for both manual and sprayable applications.

In one embodiment the surface of the formulation, such as of the capsule, the sphere, etc., may be treated with a coating; suitable coatings include waxes, and also sugars, pigments, proteins and fatty acids. This coating allows the release of volatile constituents or interaction with the environment or with the target organism to be regulated.

In one embodiment the volatile component may be a part of the nonpolar fluid.

In a further aspect the present invention provides a method for producing an oleogel formulation of the invention. This method comprises:
providing the nonpolar fluid;
introducing the gelator into the liquid nonpolar fluid, to give a liquid mixture containing gelator and nonpolar fluid; and
producing the oleogel formulation,
   where the volatile component or the active ingredients are added, depending on the hydrophobicity, to the nonpolar fluid, to the gelator before introduction into the liquid nonpolar fluid, or as a further solution separately.

The oleogel formulation may therefore be produced in accordance with customary methods. Corresponding methods are known to the skilled person.

In one embodiment the formulation is formed, for example, as a sphere, capsule, fiber, block, lens or film by dropletizing, extruding, or casting the liquid mixture onto a surface, and subsequent solidification by cooling.

In one embodiment the producing here is accomplished by extrusion, the skilled person being aware of corresponding extrusion methods. Extrusion takes place in particular for producing the formulation in the form of a core-shell capsule, by means of two-material extrusion, for example.

The skilled person is aware of corresponding extrusion methods. Production may also take place using a three-material die. Alternatively there may be extruding of the filling into the oleogel phase, especially when producing a multiple-core version of the formulation.

In one embodiment hollow spheres are produced by introducing a mixture with gelator and any volatile components or active ingredient present into a liquid oleogel-sol formulation. Such introduction may be, for example, extruding, or alternatively dropwise introduction, etc.

In one embodiment the method of the invention comprises subsequent cooling of the resultant formulation in the form, for example, of a sphere, such as a hollow sphere or solid sphere, capsule, more particularly core-shell capsule, fiber, block, lens or film. Illustrative embodiments have been explained in more detail above in reference to FIG. 1.

Depending on the cooling/gelling rate, it is possible on the industrial scale to produce either lenses, after dropletization onto a conveyor belt, or to produce spherical forms, in the case of higher cooling rates or longer “fall times” of the sol, in a cooling tower, for example. On the industrial scale, production may also be accomplished using single-screw or twin-screw extruders.

In one embodiment of the method of the invention, the surface of the resulting formulation based on the oleogel may be additionally given a layer which further inhibits diffusion or introduces other properties modifying the surface of the formulation. This may be accomplished, for example, by adding wax or other additives.

In a further aspect the use is provided of the formulation of the invention as a crop protection product or pesticide, which embraces, in particular, such products and pesticides comprising biocides, insecticides, fungicides, nematicides, acaricides or herbicides.

In a further embodiment the invention relates to the use of the formulation of the invention in the food industry, the cosmetic industry or the pharmaceutical industry. Accordingly the formulation may contain ingredients such as vitamins, amino acids, etc., or else active ingredients as a pharmaceutical product. In light of the possibilities for modifying the properties of the oleogel on the basis of the fractions of oleogelator and nonpolar fluid or on the basis of the type of formulation provided, as a core-shell capsule, for example, etc., it is possible to achieve controlled and target-directed release of the ingredients and component.

In one embodiment the use may be one embracing the use of different ingredients or components.

The formulation of the invention in the form of the capsule, for example, enables outstanding use as a crop protection product and pesticide by virtue of the hydrophobic properties of the surface. In view of the hydrophobic surface, there is outstanding adhesion of the capsule to leaves and other surfaces, thereby enabling application to leaves in agriculture, such as in fruit and vegetable growing, for example, without the use of additional sticking agents. The composition of the formulation of the invention, based on the biodegradable constituents, also makes the formulation suitable for the agricultural sector.

These and further advantages of the formulation of the invention are clear to the skilled person.

The invention is explained further, below, using examples, without being confined to these examples.

### Example 1

1. Introduction of 48 wt % sunflower oil+10 wt % octyldodecanol in a heat-resistant glass vessel with stirring bar
2. Dispersion of 10 wt % ethylcellulose in the mixture initially introduced
3. Heating to 140° C. with stirring at 100 rpm and holding for at least 1 hour until the ethylcellulose is fully dissolved
4. Cooling of the mixture to 100° C.
5. Addition of 2 wt % candelilla wax
6. Addition of 30 wt % volatile attractant, present in liquid form, and brief stirring
7. Dropletization of the hot sol onto an inert surface, and solidification by cooling

### Example 2

1. Production of the oleogel shell material as described in example 1—steps 1.-6.
2. Production of the filling material:
   2.a Introduction of 89.8 wt % water in a glass vessel with stirring bar
   2.b Addition of 0.2 wt % methylcellulose with stirring, and continuation of stirring of the mixture until the methylcellulose is fully dissolved
   2.c Addition of 10 wt % sorbitol and stirring until dissolution is complete
3. Extrusion of the filling into the oleogel sol which is at 100° C.
4. After a few seconds, the hollow spheres can be withdrawn from the oleogel sol, and the oleogel shell formed, with the filling contained in it, can be left to solidify completely on an inert surface.

Following the production (and optional storage) of the oleogel formulations, the presence and the delivery rate of the volatiles formulated therein is analyzed by means of thermodesorption-coupled gas chromatography and (thermo)gravimetry. The analysis of the formulations containing volatile semiochemicals and without such chemicals, and also an unformulated semiochemical, showed clearly that at elevated temperatures the volatile semiochemical is released with a time delay. It was also found that the semiochemical is present substantially completely in the oleogel formulation.

It was additionally possible to show what effect there is on the release of the formulated volatile by adding different concentrations of wax in the ethylcellulose-oleogel formulation. After just 177 hours, more than 80 wt % of the active volatile component has evaporated from an ethylcellulose-oleogel formulation without wax (WO); the addition of 2 wt % of candelilla wax delays the release, and even after 177 hours, still more than 80 wt % of the amount of active ingredient originally used is still present in the formulation. A variant W4 as well, containing 4 wt % of candelilla wax, showed the release-modifying effects of the wax.

1. An oleogel formulation, comprising:
0.1-20 wt % of oleogelator;
at least 0.1 wt % of at least one active ingredient or at least one volatile component; and
at least one nonpolar fluid up to 100 wt %,
where the at least one volatile component may be part of the at least one nonpolar fluid.

2. (canceled)

3. The oleogel formulation of claim 1 wherein the oleoregulator comprises at least one alkylcellulose.

4. The oleogel formulation of claim 1 wherein the at least one nonpolar solvent comprises at least one component selected from vegetable, animal, microbial, and mineral oils.

5. The oleogel formulation of claim 1 wherein the oleogelator is a combination of an alkylcellulose and a vegetable wax wherein the alkylcellose is different from the vegetable wax.

6. The oleogel formulation of claim 5, where the combination of the alkylcellose and the vegetable wax is a combination of ethylcellulose and candelilla wax.

7. The oleogel formulation of claim 1 further comprising at least one adjuvant selected from particulate solids for delayed release, fillers, UV protectants, adsorbers, density modifiers, temperature-dependently crystalline, and polymorphous substances.

8. The oleogel formulation of claim 1 wherein the at least one volatile component or the at least one active ingredient is a hydrophilic component or a hydrophilic active ingredient.

9. The oleogel formulation of claim 1 wherein the at least one active ingredient or at least one volatile component is or comprises one or more hydrophobic volatile semiochemicals in a matrix of the oleogel.

10. The oleogel formulation of claim 1 formed as a core-shell capsule.

11. The oleogel formulation of claim 1 formulated as capsules or granules having hydrophilic regions surrounded by hydrophobic oleogel.

12. The oleogel formulation of claim 1 is configured with hydrophilic active ingredients and/or hydrophilic volatile components in at least one hydrophilic region and hydrophobic active ingredients and/or hydrophobic volatile components in the hydrophobic region.

13. The oleogel formulation of claim 1 is configured as a matrix in a form selected from the group consisting of a solid sphere, a hollow sphere, a fiber, a block, a lens, and a film.

14. The oleogel formulation of claim 10 wherein the core-shell capsule comprises a liquid hydrophilic core.

15. The oleogel formulation of claim 1 configured as a paste.

16. A method for producing an oleogel formulation of claim 1, comprising:
providing the at least one nonpolar fluid;
introducing at least one gelator into the at least one nonpolar fluid to produce a liquid mixture containing the at least one gelator and the at least one nonpolar fluid; and
producing the oleogel formulation from the liquid mixture,
wherein the at least one volatile component or the at least one active ingredient are added, depending on hydrophobicity, to the at least one nonpolar fluid, or to the at least one gelator before introduction into the at least one liquid nonpolar fluid, or as a separate solution.

17. The method of claim 16, further comprising the formation of a sphere, capsule, fiber, block, lens or film by dropletizing, extruding, or casting the liquid mixture onto a surface, and subsequent solidification by cooling.

18. The method of claim 16 wherein the liquid mixture is extruded into a liquid oleogel-sol formulation, and producing hollow spheres from the combination of the liquid mixture and the liquid oleogel-sol formulation.

19. The method of claim 16 where producing takes place by extrusion, the oleogel formulation being produced as a core-shell capsule by two- or three-material extrusion.

20. A method of using the oleogel formulation of claim 1 as a crop protection product or pesticide, comprising applying the oleogel formulation to a crop.

21. The oleogel formulation of claim 3 wherein the at least one alkyl cellulose is selected from the group consisting of ethylcellulose, wax, sterols, fatty alcohols, fatty acids, wax acid, wax esters, fatty acid esters, isoprene derivatives, triglycerides, diglycerides, monoglycerides, lecithins, and stearates.

22. The oleogel formulation of claim 9 wherein the hydrophobic volatile semiochemicals are selected from pheromones and allelochemicals.
